(19) United States
(12) Patent Application Publication
(10) Pub. No.: US 2007/0225809 A1
(43) Pub. Date: Sep. 27, 2007

(54) System and device for filling a human implantable container with a filler material

(76) Inventor: Charles D. Ray, Santa Barbara, CA (US)

Correspondence Address: DICKE, BILLIG & CZAJA, FIFTH STREET TOWERS, 100 SOUTH FIFTH STREET, SUITE 2250, MINNEAPOLIS, MN, 55402, US

(21) Appl. No.: 11/390,515

(22) Filed: Mar. 27, 2006

(51) Int. Cl. A61F 2/44

(52) U.S. Cl. 623/17.12

(57) ABSTRACT

A small, simple user-operated device for filling an implantable container or space inside a body structure or a void is disclosed. The device includes a delivery tube defining a load port at a proximal end and an ejection port at a distal end, an auger rod disposed within the delivery tube and extending from the load port to the ejection port, and means for rotating the auger rod. In this regard, rotation of the auger rod transports implantable filler material away from the load port and through the delivery tube and out of the ejection port to fill the container.

### FIELD OF THE INVENTION

Aspects of the present invention relate to a system and a device useful in filling a human implant with a filling (particulate or viscous liquid) material. More specifically, aspects of the present invention relate to an augering device useful for filling a human implantable container (especially a flexible or highly flexible container) inserted into a prepared body cavity, for example a jacket of an artificial disc nucleus.

### BACKGROUND

Implantable devices having a cavity-defining container, such as prosthetic disc nucleus jackets or balloons inserted to treat vertebral collapse fractures, have been disclosed in prior art. One example of an implantable disc prosthesis is generally made of a hygroscopic polymer pellet surrounded by a retaining jacket as taught by Ray, et al. in U.S. Pat. Nos. 4,722,287; 4,904,260 and 5,674,295 A wide variety of other cavity-type implants are used to achieve the filling of bone voids or bone fusions.

Many of the above-mentioned implants insert the filler component into the container prior to implant. For example, some prosthetic disc nucleus devices presently utilize a pre-filled jacket for implantation into the surgically prepared disc space. In other surgical applications, however, it is of benefit to implant the container prior to insertion of the filler material. In general terms, a container (rigid or flexible) defining an internal cavity is implanted in the patient at the desired implantation site, followed by filling (partial or complete) of the cavity with an appropriate material. For example, Assell et al., U.S. Pat. No. 6,022,376, teaches filling the flexible container (or jacket) cavity following implantation using various solutions and suspensions of particulate materials in conjunction with a small diameter tube or needle. However, particulate materials require a fluidizing or carrying agent since they do not pass easily through small diameter tubes or needles, as would be required to fill an already implanted device container (e.g., jacket). Further, the particles are too coherent and viscous to be injected without a carrier. The carrier material presents additional problems regarding the total volume of the injectate and the tissue reactivity to it.

Prior art also teaches the use of various fluid substances, typically, one or two part polymeric compounds, particulate polymeric grains, or tissue particles such as bone injected or inserted under pressure inside an implanted cavity-forming container (e.g., bag or porous woven sack) that has been previously or simultaneously inserted into a prepared body site.

Two issues regarding the implantation of prostheses or other devices have nonetheless remained: (1) the need for a small formed insert, requiring a small access port in the body for ease and increased safety during insertion that when filled becomes substantially larger and (2) an improved conformity of the inserted prosthesis or other device to the usually irregular, evacuated bodily site.

With the above background in mind, improvements to, and advancement of filling of a previously implanted container (jacket, pocket or sack) will be welcomed by surgical developers of implants and by the surgeons utilizing them for patient benefit.

### SUMMARY

One aspect of the present invention provides a device for forcing a filler material into a human implantable, biocompatible container. The device includes a delivery tube defining a load port at a proximal end and an ejection port at a distal end, an auger rod disposed within the delivery tube and extending from the load port to the ejection port, and means for rotating the auger rod. In this regard and during use, rotation of the auger rod transports filler material away from the load port, through the delivery tube and out of the ejection port.

Another aspect of the present invention provides an implantation system. The system includes biocompatible, human implantable container, a filling device, and a supply of filler material. The container is expandable and defines a cavity and a cuffed fill port. The filling device is removably coupled to the fill port of the implantable container. In this regard, the filling device includes a delivery tube defining a load port at a proximal end and an ejection port at a distal end, an auger rod disposed within the delivery tube and extending from the load port to the ejection port, and means for rotating the auger rod. During use of the system, rotation of the auger rod transports the filler material introduced to the load port through the delivery tube and out of the ejection port into the fill port of the implantable container.

Yet another aspect of the present invention provides a method of implanting an implantable device into a human patient. The method includes implanting an implantable, biocompatible container defining a cuffed fill port within the patient. The method additionally includes fluidly coupling an ejection port of a delivery tube into the cuffed fill port. Implantable filler material is placed into a load port of the delivery tube. An auger rod within the delivery tube is then rotated to transport the filler material from the load port to the ejection port and force the filler material into the implanted container.

In one embodiment, a small diameter auger tube to move the particulate filler material by mechanical means and not by applied hydraulic pressure is provided. The filling device includes a tube of slightly greater diameter than that of the particles and having a pitch determined by experimentation that properly moves the particular filler material using manual means. The tubular unit or filling device is initially attached to an implantable, biocompatible container (e.g., flexible empty bag or jacket) using a firmly attached drawstring. The container, attached to the tube, is implanted into a bodily site of the patient and manual rotation of the auger moves or forces the particulate filler material inside the container. The augering force is delivered manually using suitable means. In some embodiments, manual feedback, plus fluoroscopic visualization of the surgical site, informs the surgeon as to the appropriateness of the container's filling and conformity. The mass of the injected filler material (e.g., particles) forms coalescence after placement that inhibits loss of the particles into the surrounding tissue space. The proper positioning of the filling and filled container (e.g., jacket or sack) can be confirmed using x-ray images, when the container has simple radio-opaque markers attached at either or both of its ends.

Another aspect of the present invention provides an adjunctive reservoir with larger capacity providing the user with a large, continuous flow of filler material particles into the implantable container. The method may further include the addition of medications to be swept with the particles inside the container for various indications. The novel device can be adapted to the extrusion of tissue particles filler materials such as bone, bone substitutes, collagen or connective tissue components or particularized therapeutic materials to fill appropriate body cavities, natural, pathological or surgically created.

Still another aspect of the novel particle-injecting system provides a drawstring to be tightened around the tubular auger as it is removed. In this regard, the escape of the filler material particles into the surrounding tissues is prevented. Thus, when sufficient volume has been placed, simple remote means permit firmly tying or sealing of the container access port. Additionally, in some embodiments, the final, filled container is configured to permit or inhibit the ingrowth of surrounding tissue, as desired for a particular surgical outcome. Further, the outer and inner auger tubes may be rigid or bendable to accommodate the method of insertion and extrusion of the injectable filler material. If the novel method does not suffice, an initial pre-filled container device and method may be employed.

The present invention solves the problems of particulate injection into an implanted, unfilled or partially-filled container placed or implanted into the patient (e.g., prepared nucleus cavity of a disc space or other body cavity). In one embodiment, the container is removably attached to a delivery tube defining an outside diameter of between approximately 1 mm to 10 mm, preferably the outside diameter is between 4 mm to 6 mm, although other diameters for the delivery tube are also acceptable. The auger extrudes the viscous particulate filler material (e.g., hygroscopic material or fluid) into the container filling it under sufficient pressure to lift the space and cause the flexible container to conform to the evacuated cavity. In some embodiments, further hydration of a particulate hygroscopic medium filler material additionally expands the device to a volume and function as desired or to a volume reasonably similar to that of a normal tissue complex.

The diameter of the auger is of suitable diameter to accommodate the dimensions of the filler material (e.g., particulate polymeric material) or viscosity of the filler material (e.g., fluidized medium). In some embodiments, the determination of suitable diameter of the auger tube is determined by prior bench testing and through the study of the implant site (e.g., cadaveric human vertebral segments).

The novel device may be constructed of suitable polymers to render it sterilizeable (by gas or radiation) and disposable after single use.

### BRIEF DESCRIPTION OF THE DRAWINGS

Embodiments of the invention are better understood with reference to the following drawings. The elements of the drawings are not necessarily to scale relative to each other. Like reference numerals designate corresponding similar parts.

FIG. 1 illustrates a perspective view of an implantation system according to one embodiment of the present invention.

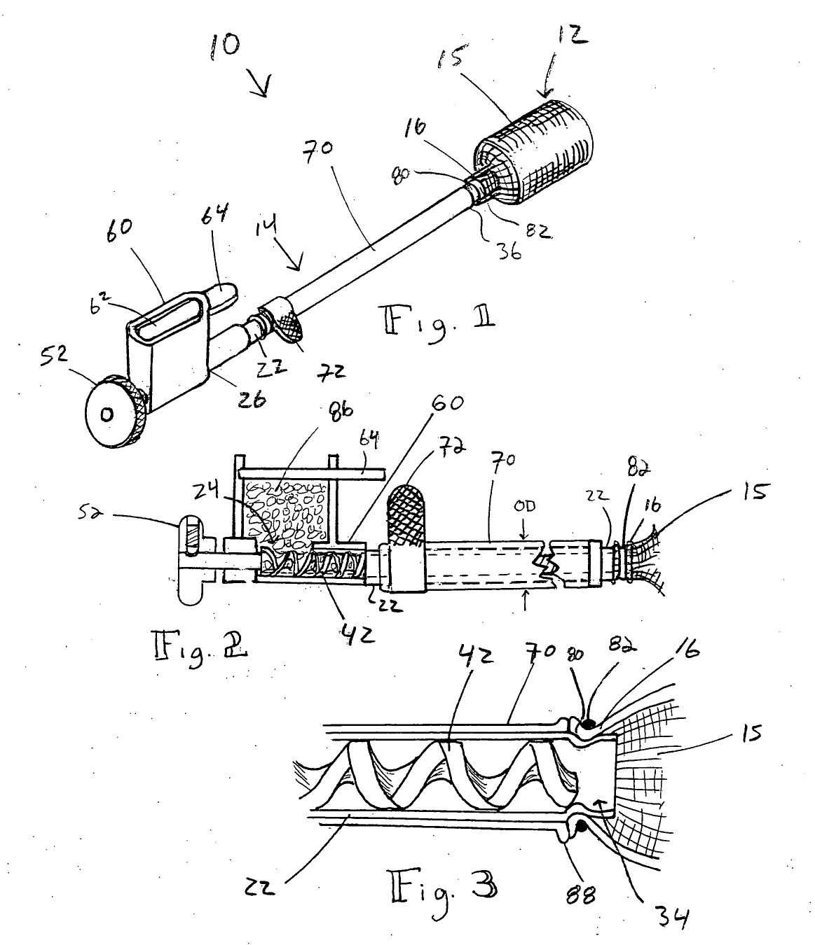

In this regard, FIG. 1 is a diagrammatic tangential plan view of the auger tube showing the filler material particle supply reservoir at one end and the removable attachment of the human implantable, biocompatible housing with associated means to close same following the filling in situ, at the other end. A manual dial-like crank or other similar means applies the augering force needed to move the filler or particulate material along inside the tube into the implantable container in accordance with the present invention. In one embodiment, the container has radio-opaque markers in the extreme ends for x-ray visualization.

FIG. 2 illustrates a cross-sectional view of the system of FIG. 1, according to one embodiment of the present invention.

In this regard, FIG. 2 is a diagrammatic cross-section of the augering device, the filler material supply reservoir and the detachable container, with closure drawstring. In this regard, in one embodiment the novel device is a particulate materials or viscous fluid mobilizer or injector. During use, the desired filler substance(s) may be augered into the empty container in order to fill a prepared bodily site (e.g., cavity) providing a much broader selection of substances than can be injected, as needed by the surgeon-user.

FIG. 3 illustrates an enlarged, cross-sectional view of a portion of the distal end of the system of FIGS. 1 and 2. In this regard, the augering device illustrates the auger, the inner carrier/delivery tube and an outer tube used to separate the filled container into the desired body space. Also shown is an affixed drawstring used to close the container after separation from the inner tube.

For one skilled in the art, other auger and detachment-jacket sealing designs may be substituted without changing the intent and performance of the invention.

### DETAILED DESCRIPTION

In the following Detailed Description, reference is made to the accompanying drawings which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments of the present invention can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

The Figures illustrate diagrammatically embodiments of the invention where particulate filler material, such as bone chips, may be carried into a body cavity or into a formed container placed within a body cavity.

FIG. 1 illustrates a perspective view of an implantation system 10 according to one embodiment of the present invention. The system 10 includes a human implantable, biocompatible container 12 and a filling device 14. The container 12 defines an internal cavity 15 (referenced generally) and a cuffed fill port 16. The filling device 14 is removably coupled to the fill port 16. In this regard, the filling device 14 includes a delivery tube 22 defining a load port 24 (FIG. 2) at a proximal end 26 and an ejection port 34 (FIG. 3) at a distal end 36, an auger rod 42 co-axially disposed within the delivery tube 22 and extending from the load port 24 to the ejection port 34, and a crank or other actuation means 52 configured to rotate the auger rod 42.

In one embodiment, a reservoir 60 is coupled to the proximal end 26 of the delivery tube 22, where the reservoir 60 is in fluid communication with the load port 24 (FIG. 2) and configured for containing the filler material (shown at 86 in FIG. 2). In one embodiment, the reservoir 60 defines a reservoir opening 62 and a cover 64 removably disposed over the reservoir opening 62.

In one embodiment, an outer tube 70 is slidably disposed over the delivery tube 22, and the outer tube 70 defines a radially extending tab 72.

In one embodiment, the delivery tube 22 defines a recess 80 extending circumferentially about the distal end 36. In one embodiment, the delivery tube 22 defines an outside diameter O.D. (FIG. 2) of between approximately 1-10 mm, and preferably the O.D. is between 4-6 mm.

The implantable container 12 can assume a wide variety of forms, and is selected in accordance with the particular procedure being performed. The container 12 is formed of biocompatible material(s) appropriate for human implantation. The container 12 can be porous or fluid impermeable, and can be rigid, semi-flexible or flexible. The system 10 is particularly useful with a flexible or highly flexible container 12 configuration, whereby the container 12 can be forced to a reduced size or volume for easier implantation, followed by expansion to a desired size/volume when filled (partially or completely) with the filler material 86. For example, the container 12 can be a woven jacket or sack. Along these same lines, the fill port 16 can be defined by the container 12 in a wide variety of manners, and is generally characterized as providing a closeable, reduced size opening or inlet fluidly connected to the cavity 15. For example, in one embodiment, the container 12 further includes a string or wire 82 circumferentially surrounding the fill port 16 in a manner akin to a purse string. With this one construction, the string 82 can be tensioned and tied to close (and seal, in some embodiments) the fill port 16. Alternatively, a wide variety of other closure assemblies can be employed.

While the system 10 has been described as including the implantable container 12, in alternative embodiments, the filling device 14 can be employed to deliver desired particulate or other filler material directly into a bodily site of the patient, such that the implantable housing 12 can be eliminated.

With reference to FIGS. 1-3, the assembled system 10 is shown with the fixedly removable, fillable container 12 removably coupled to the distal end 36 of the delivery tube 22. The container 12 is attached to the tube 22 via the cuff 16 and a drawstring 82. The reservoir 60 is attached to the tube structures to hold and dispense the filler material to be inserted into the container 12 or bodily space. Markings (not shown) inside the reservoir 60 indicate contained and dispensed material volumes. The closable lid 64 protects the contained filler material 86. The reservoir 60 is affixed to the delivery tube 22. An actuation means, such as the crank 52, is provided to manually rotate the delivery auger rod 42. The sliding outer tube 70 in one embodiment is provided with a thumb tab 72 that allows the sliding tube 70 to be forced against the container cuff 16. Alternatively, other means for rotation of the auger or for sliding the outer tube can be employed.

FIG. 2 illustrates a partial cross-sectional view of the system 10. The actuation means 52 is removably attached to a parallel rod portion of the auger rod 42. In one embodiment, the spiral 42 closely fits inside the delivery tube 22 of the filling device 14. The implantable filler material 86 (e.g., bone chips, etc.) to be dispensed by the device 14 is held inside the reservoir 60. In one embodiment, the actuation means 52 is removably coupled to from the auger 42 to facilitate cleaning. Rotation of the manual means 52 causes the auger rod 42 to move the filler material 86 along the delivery tube 22 and into the container 12 or sack attached at the distal end 36 of the device 14.

FIG. 3 illustrates details of components at the distal end 36 of the filling device 14. The wall and the cuffed fill port 16 of the container 12 removably passes beneath the purse string 82. The delivery tube 22 has a formed constriction, or groove 80, configured to removably receive the container 12/cuffed fill port 16 and the purse string 82. Where the fill port 16 is of a differing design (e.g., that may or may not include the string 82 as a closure mechanism), the delivery tube 22 will assume a corresponding structure for mated assembly. Regardless, the delivery tube 22 is closely fitted to the outer diameter of the auger rod 42 and the inner diameter of the outer tube 70. The delivery tube 22 is the conduit for the implantable filler material 86 being forced into the cavity 15 of the removable container 12. The end of the outer tube 70 defines a blunt end 88, where the blunt end 88 acts as a pusher to remove the cuffed fill port 16 of the container 12 and the purse string 82 following satisfactory filling of the container 12. In one embodiment, the action to displace the container 12 from the delivery tube 22, using the blunt end 88 is operated by the thumb tab 72 of FIGS. 1 and 2. On pushing off the container 12, the purse string 82 is suitably tightened, preventing escape of the contained material from the cavity 15 through the cuffed fill port 16.

The injected filler material (e.g., particulates) 86 may be hygroscopic, expanding further after injection into the container 12. Where the container 12 is flexible or highly flexible, the injection or insertion pressure preferably provides sufficient lifting power to fill the relatively flattened container 12 (as initially implanted) and elevate the evacuated space to a desired configuration under load. For example, but in no way limiting, when applied to the human intervertebral disc space, the material injection pressure generally will be about 2-4 atmospheres, 30 to 60 psi, but may be more or less depending upon the surgical situation. The apparent viscosity of the filler material 86 inhibits free injection of thickened (or most particulate materials) through a tube. Embodiments of the present invention overcome this situation and enable filling of a wide range of viscosities of implantable filler materials 86.

Alternative embodiments having additional use include polymer tubing and an overall construction suitable for sterilization and single use disposability of the novel unit.

### Method And Example of Use

With reference to FIGS. 1-3, the container 12 (e.g., porous jacket or sack) is removably attached to the distal end of the delivery tube 22 and together with the outer tube 70; its distal end is inserted into the prepared surgical site (e.g., body cavity to be filled). In one embodiment, x-ray visible radio-opaque markers are included in the container 12. Proper location of the container 12 can be determined using a fluoroscopic x-ray unit. The particles to be implanted, with or without an accompanying carrier fluid are pre-measured and loaded into the reservoir 60 or hopper and the protective lid 64 closed. Manual turning of the auger rod 42 moves the pre-measured volume of filler material 86 particles into the pre-determined body site (e.g., into the container 12). When the appropriate, pre-determined volume of the particulate filler material 86 has been injected, the container 12 is maneuvered off the end of the delivery tube 22 such as by using force applied by the thumb tab on the outer tube 70. The drawstring 82 (or other closure device) is tightened during the removal, closing the housing 12 neck/fill port 16 to prevent any loss of filler material particles into the surrounding tissues.

### Advantages

The invention provides the ability to introduce particulate filler material or fluids of high viscosity that ordinarily cannot be injected through a smaller tube or needle. This ability is accomplished using a small spiral augering means. The method is simple to apply and a measured quantity of particulate or viscous injectate can be dispensed easily into a confining, biocompatible container, or inserted into a prepared or diseased body space or cavity. The injection or delivery of the material is under control by the user at all times. The device and method can be used with or without adjunctive carrier fluid, that is, with small particulates alone or accompanied by a fluid carrier, as required by the particular user. The user employing the device simply fills the reservoir, or hopper with the injectate and moves it manually into the desired space, the rate and position of the collection of filler material particles into a mass can be continuously monitored using an x-ray fluoroscope and the volume injected as well as the location of the filling jacket or sack can be adjusted as desired. At completion of the filling of the container, a simple procedure disengages the housing from the tubular auger and a drawstring (or other closure device) is tightly pulled. The ends of the strings (or other closure device) are tied to prevent escape of the injected substance.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

While the preferred embodiments of the invention have been described, it should be understood that various changes, adaptations and modifications may be made therein by those skilled in the art without departing from the scope of the invention.

The application of packing the particulate or viscous substance inside the human implantable, biocompatible container is devoid of any undesirable side effect such as overfilling or leakage of the substance into neighboring tissues by indirect observation using x-ray fluoroscopy, readily available in operating rooms, by monitoring any changes in injection effort or by changes in the rate of injection of the substance. These latter effects are determined by the user's tactile senses as the auger is turned by hand. The effects of such safety are immediate and continuous. With the device disclosed herein, the surgeon using the hand-held device can advantageously control and direct the location of volumetric filling of the container and can adjust the rate and quantity of substance as indicated. The auger can be constructed of such as surgical grade stainless steel or an appropriate firm polymer, with the hopper fixably attached or removable. The device can be reusable or disposable. It can be used to insert or inject substances or viscous fluids such as bone particles, collagen, etc., to fill body defects. Further medications may be added to the injectate as desired. The reservoir or hopper may be attached to the auger assembly in a variety of ways. Likewise, the detachment of the container can be achieved in a variety of ways so that no injected substance escapes during detachment and tying of the drawstring or other closure device.

Preferred means to deliver the substance, attach and remove the container (e.g., jacket or sack) are disclosed here although persons skilled in the mechanical arts can adapt the concept to a variety of means to cause desirable insertion of a particulate or viscous substance into a receiving space.

What is claimed is:

1. A human implantation system comprising:
a human implantable, biocompatible container defining a cavity and a cuffed fill port; and
a filling device removably coupled to the fill port of the container, the filling device including:
a delivery tube defining a load port at a proximal end and an ejection port at a distal end,
an auger rod disposed within the delivery tube and extending from the load port to the ejection port,
actuation means for rotating the auger rod;
wherein rotation of the auger rod transports filler material introduced to the load port through the delivery tube and out of the ejection port into the fill port of the container.

2. The implantation system of claim 1, wherein the delivery tube defines a recess extending circumferentially about the distal end, the recess configured to receive the cuffed fill port of the container.

3. The implantation system of claim 1, further comprising:
an outer tube slidably disposed over the delivery tube, the outer tube configured to slide against and displace the cuffed fill port of the container once filled with the filler material.

4. The implantation system of claim 3, wherein the outer tube includes a tab radially extending from a proximal end of the outer tube, the tab configured to provide a means for sliding the outer tube relative to the delivery tube.

5. The implantation system of claim 1, wherein the actuation means is a crank.

6. The implantation system of claim 1, further comprising:
a reservoir coupled to the proximal end of the delivery tube, the reservoir in fluid communication with the load port and configured for introducing filler material to the load port.

7. The implantation system of claim 1, further comprising:
a supply of filler material including at least one of bone chips, collagen, and hydroscopic polymer.

8. The implantation system of claim 1, wherein the container is flexible.

9. A method of implanting an implantable device to a human patient, the method comprising:
implanting a biocompatible container at a desired bodily site of the patient, the container defining a cavity and a cuffed fill port;
providing a filling device including a delivery tube coaxially disposed about an auger rod;
fluidly coupling an ejection port of the delivery tube to the cuffed fill port;
introducing filler material to a load port of the delivery tube; and
rotating the auger rod within the delivery tube to force the filler material into the container.

10. The method of claim 9, wherein fluidly coupling the ejection port the cuffed fill port includes securing the delivery tube to the cuffed fill port by retaining the cuffed fill port within a recess defined around a circumference of the delivery tube adjacent to the ejection port.

11. The method of claim 9, wherein rotating the auger rod includes transporting particulate filler material through the delivery tube and into the housing.

12. The method of claim 9, wherein rotating the auger rod within the delivery tube comprises transporting viscous filler material into the housing.

13. The method of claim 9, further comprising:
after forcing the filler material into the container, removing the container from the delivery tube by sliding an outer tube over the delivery tube to disengage the cuffed fill port from the delivery tube.

14. The method of claim 13, wherein sliding the outer tube includes engaging a tab extending from the outer tube in translating the outer tube over the delivery tube.

15. The method of claim 9, wherein the ejection port is fluidly coupled to the fill port prior to implanting the container within the patient.

16. The method of claim 9, wherein the container is flexible, and further wherein implanting the container includes forcing the container to a reduced size prior to implantation within the patient.

17. A filling device for delivering a filler material into a human implantable, biocompatible container, the device comprising:
a delivery tube defining a load port at a proximal end and an ejection port at a distal end;
an auger rod disposed within the delivery tube and extending from the load port to the ejection port; and
means for rotating the auger rod;
wherein rotation of the auger rod transports filling material away from the load port and through the delivery tube and out of the ejection port.

18. The device of claim 17, further comprising:
an outer tube slidably disposed over the delivery tube, the outer tube defining a radially extending tab.

19. The device of claim 17, wherein the delivery tube defines a recess extending circumferentially about the distal end.

20. The device of claim 17, wherein the delivery tube defines an outside diameter of between approximately 1-10 mm.
